(19) United States
(12) Patent Application Publication
(10) Pub. No.: US 2024/0382354 A1
(43) Pub. Date: Nov. 21, 2024

(54) Cover Nonwoven

(71) Applicant: Sandler AG, Schwarzenbach (DE)

(72) Inventors: Uwe BERNHUBER, Hof (DE); Jessica ZEITLER, Hof (DE)

(21) Appl. No.: 18/632,011

(22) Filed: Apr. 10, 2024

(30) Foreign Application Priority Data
Apr. 13, 2023 (DE) 202023000825.5

(51) Int. Cl. A61F 13/551

(52) U.S. Cl. CPC A61F 13/5516

(57) ABSTRACT

The invention relates to a cover nonwoven for feminine hygiene products which consists of 100% cellulose fibers, is mechanically consolidated and the surface is mechanically smoothed.

### CROSS-REFERENCE TO RELATED APPLICATION

This application claims the benefit of priority to German Patent Appl. No. 202023000825.5 filed Apr. 13, 2023, the disclosure of which is hereby incorporated by reference in its entirety.

### BACKGROUND

The demand for sustainability in the manufacture of personal hygiene articles is reflected in the demand for sustainability of the individual components. The SUPD (Single Use Plastic Directive) also poses a challenge for manufacturers to use materials without plastic content. There is currently a labeling requirement for single-use plastic products and a tax on single-use products with plastic content will be introduced.

The present invention relates to cover nonwovens for feminine hygiene articles, for example as topsheet materials for panty liners or in particular enveloping nonwovens for tampons, which are used for feminine hygiene.

The state of the art is explained using menstrual tampons as a representative but not limiting example.

Tampons of the generic type consist of
a cover nonwoven,
an absorbent core and
a return thread.

In this context, the cover nonwoven has several functions: for example, it must ensure easy insertion of the tampon, must allow body fluids to pass through to the absorbent core and must retain any fibers protruding from the absorbent core.

In the manufacture of tampons, a card sliver made of viscose or blends of viscose and cotton is usually bonded to a cover nonwoven made of synthetic fibers using a heat-sealing method. For example, nonwoven constructions with bi-component staple fibers made of polyethylene in the sheath and polypropylene or polyethylene terephthalate in the core are used. During the so-called sealing, the bonding of the cover nonwoven with the absorbent core, the low-melting sheath component is used for gluing. For optimum insertion of the tampon, a particularly smooth cover nonwoven is also preferred. Such products, described in DE 19753665 C2, exhibit high gliding properties.

The disadvantage of thermally bonded materials as tampon covers is that no 100% sustainable raw material can be used, as the fibers are bonded using heat. For this purpose, fossil raw materials must be reverted to. Another disadvantage of these materials is their stiff, synthetic feel.

In EP 3943054 B1 and in EP 2776619 B1, nonwoven fabrics are described that consist of 100% biodegradable, sustainable fibers. These nonwoven fabrics can be used as a cover nonwoven for a menstrual tampon. It has already been recognized here that a cover nonwoven made of 100% sustainable fibers does not have good gliding properties. EP 22776619 B1 therefore provides for a chemical treatment of the fibers used to improve the surface smoothness. An additional chemical treatment of nonwoven fabrics as a sheath for a tampon core is not desired by the end user.

The object of the present invention is therefore to provide a cover nonwoven made of cellulosic fibers for feminine hygiene products, which consists of 100% sustainable fibers and has a smooth and even surface structure.

The problem is solved with the aid of the features of claims 1-6.

### BRIEF DESCRIPTION OF THE DRAWINGS

FIG. 1 depicts an exemplary test set up to determine the static and dynamic coefficient of friction of cover nonwoven of present disclosure.

FIG. 2 depicts an example of the force curve over the test length.

### DETAILED DESCRIPTION

A cover nonwoven manufactured according to the invention is made from 100% cellulosic fibers by carding technology and mechanically consolidated by needling, preferably by the spunlace method. The term spunlace refers to hydroentanglement.

Due to its thickness and uneven structure, a purely cellulose spunlace nonwoven is only suitable for a tampon cover to a limited extent. In order to influence the functionality of a spunlace material accordingly, the material is subsequently calendered.

By means of calendering, the spunlace nonwoven is smoothed and its thickness reduced. A nonwoven treated in this way can then be used as a cover nonwoven, e.g. as an enveloping nonwoven for a tampon or as a topsheet for panty liners or similar.

Thus, 100% sustainable fiber material can be used for manufacture and yet the properties of a thermally consolidated material can be simulated.

The following test methods were used for the examination:
Basis weight according to WSP 130.1, indicated in g/m²
Thickness according to WSP 120.6, measuring pressure 0.5 kPa, indicated in mm
(The WSP methods are available from EDANA, B-1160 Brussels, Belgium)

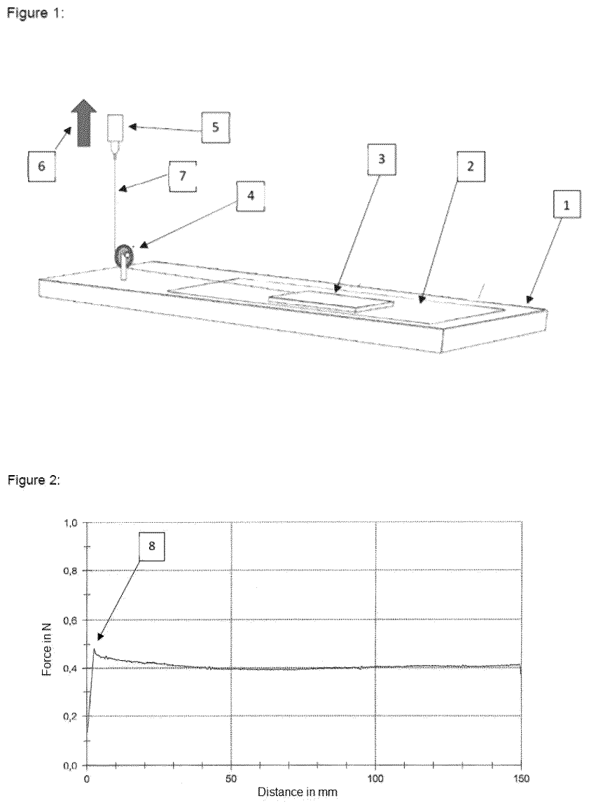

Coefficient of friction in accordance with DIN EN ISO8295-10/2004, indicated as a dimensionless quotient of frictional force (in N) and normal force (1.96N) exerted by the test slide (3). The test setup can be seen in FIG. 1. In the context of the present invention, the static and dynamic coefficient of friction was determined as follows.

| Dimensions of the slide (3): | Square metal disk with edge length | |
|---|---|---|
| | 63.5 mm *63.5 mm | |
| Weight of the slide (3): | 200 | g |
| Surface of the slide (3): | Steel, polished | |
| Test speed: | 127 | mm/min |
| Test length: | 150 | mm |
| Sample size: | 150*300 | mm |

In deviation from DIN EN ISO 8295, the slide (3) is set in motion, the test table (1) is not. For this purpose, the sample (2) is fixed on the test table (1) without creases and the slide (3) is placed at the starting point of the test. The slide (3) is connected to the load cell (5) by means of a non-stretch polyamide cord (7) via the low-friction deflection pulley (4). The slide (3) is pulled in the direction of movement (6) by moving the load cell (6). The slide (3) is pulled over the entire test length of 150 mm at a constant test speed of 127 mm/min. The force applied in each case is recorded with the load cell (5).

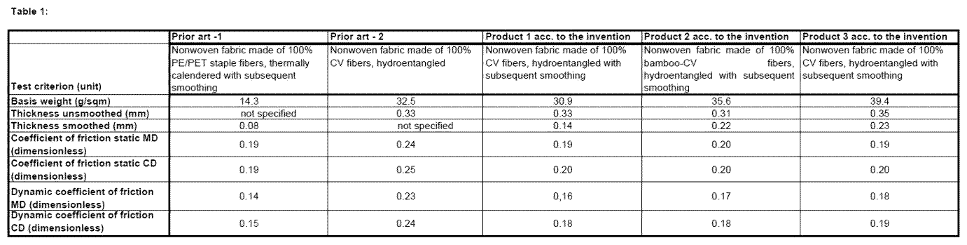

FIG. 2 shows an example of the force curve over the test length. The Y-axis shows the force applied to the load cell (5) in N, the X-axis shows the test length in mm.

The maximum force (8) observed within the first 10 mm of the test length is recorded as the static frictional force Fs. The average force that results for the movements of the slide on the test section after the first 10 mm up to the end of the test section at 150 mm is referred to as the dynamic frictional force Fd.

The respective coefficients of friction are then calculated in accordance with EN ISO 8295:2004 using the formulas from sections 3.4.1 (static coefficient of friction) and 3.4.2 (dynamic coefficient of friction).

Sustainable raw materials or sustainable fiber materials are defined in the context of the present invention as cellulosic fibers made from regeneratively obtained cellulose, such as viscose fibers. They consist of polymer chains of the macromolecule cellulose, wherein the polymer chains usually have an average degree of polymerization of 200-600. Commercially available viscose fibers for the textile industry usually also have hydrophilic properties. The sinking time is usually <1 minute. Cellulosic fibers with a fineness of between 1.3-2.9 dtex in bale form are used to manufacture the cover nonwovens according to the invention. These cellulosic fibers are commercially available, for example, from the companies Lenzing AG or Kelheim.

The following explanations refer to hydroentangled nonwoven fabrics; other mechanical consolidation methods are conceivable.

Drying method: The manufacture of the base nonwoven fabric for cover nonwovens according to the invention can be carried out using a drying method described in the book “Vliesstoffe” (nonwoven fabrics), published by Wiley VCH, 2^{nd} edition, 2012. Preferred according to the invention, but without being limited to it, is the carding process using staple fibers.

Hydroentanglement: refers to a consolidation process for cover sheets according to the invention. The basic techniques are described in the book “Vliesstoffe” (nonwoven fabrics), published by Wiley VCH, 2^{nd} edition, 2012, pages 340-359.

Calendering: a standard textile calender from the textile finishing industry is used for calendering. The calender usually consists of two rotating rollers that are pressed together with pressure and are not heated.

A cover nonwoven according to the invention consists of 100% cellulose fibers. Such a cover nonwoven can be manufactured as follows, without being limited to this:
Opening and blending the fiber bales of cellulosic fibers
Forming an unconsolidated fiber pile, e.g. by carding
Consolidating the fiber pile by means of mechanical consolidation using needles or water jets, for example
Drying the nonwoven fabric.
Smoothing the surface of the nonwoven fabric/compaction by calender passage

The excess moisture, which is introduced into the nonwoven by the consolidation process using the consolidation method of water jet, is removed by a subsequent drying process, so that the nonwoven fabric manufactured in this way according to the invention has a residual moisture content of 7 to 13% by weight.

Essential to the invention is a subsequent calender passage of the previously formed nonwoven fabric. In this process, the nonwoven is guided through the gap of a calender, e.g. a two-roller calender with steel/steel or steel/rubber rollers. The rollers thereby press against each other with a line pressure of 125 daN/cm.

Depending on the construction of the hygiene article, the basis weight of a nonwoven fabric manufactured in this way is in the range of 30 to 50 g/m², preferably 30 to 40 g/m².

The following data can be read from Table 1:

A nonwoven-1 according to the prior art of DE 19753665 C2, formed from 100% bi-component fiber with polyester core and polyethylene sheath, was fully consolidated with pressure and heat after fiber pile formation. The nonwoven has a paper-like feel, the static coefficient of friction is 0.19 in both orientations in the transverse direction (CD) and longitudinal direction (MD). The dynamic coefficient of friction is 0.14 in MD and 0.15 in CD. The coefficients of friction determined represent the generally accepted standard for cover nonwovens used as enveloping nonwovens for menstrual tampons. Due to the fibers used, such a nonwoven is not biodegradable and the focus on sustainable products cannot be guaranteed.

A nonwoven-2 consists of 100% viscose fibers. Compared to nonwoven-1, nonwoven-2 has a greater thickness and also significantly higher coefficients of friction, both static and dynamic. Although the nonwoven is biodegradable, its thickness and high coefficient of friction make nonwoven-2 unsuitable for the manufacturing process of menstrual tampons. The high coefficients of friction also lead to problems during the manufacture of panty liners, as the individual panty liners do not glide into the packaging without creasing.

The nonwovens 3, 4 and 5 according to the invention each use biodegradable fiber materials. Nonwovens 3 and 5 consist of viscose fibers, the cellulose of which was obtained from trees, while nonwoven 4 consists of viscose fibers, the cellulose of which was obtained from bamboo.

The nonwoven fabrics 3, 4 and 5 according to the invention were each subjected to a calendering treatment. The calendering treatment was carried out using a two-roller calender with steel/rubber rollers subjected to a line pressure of 125 daN/cm and a speed of 25 m/min.

Compared to nonwoven 2, nonwoven 3 configured according to the invention has a 58% lower thickness after calendering, the static coefficient of friction is 20% lower and the dynamic coefficient of friction is 30% lower. The hydroentangled and calendered nonwoven 3 according to the invention is thus in the range of the friction coefficients of nonwoven 1.

The nonwovens 4 and 5 according to the invention have higher nonwoven weights and were subjected to the same calendering treatment by two rollers with a line pressure of 125 daN/cm at a speed of 25 m/min. Surprisingly, it turned out that it is not the speed but the pressure that is decisive for achieving the surface smoothness.

Here, too, there is a significant reduction in the coefficients of friction, so that the static coefficient of friction is below 0.20 and the dynamic coefficient of friction below 0.18.

Nonwoven fabrics thus manufactured according to the invention are suitable as cover nonwovens for menstrual tampons but also as topsheets for panty liners, sanitary towels or the like. They combine the requirement for gliding properties, expressed as a coefficient of friction of less than 0.20, with the
Utilisation of 100% sustainable raw materials
Reduction of the uneven surface structure and improvement of the softness of the material
Improvement of fluid management with regard to the intake of fluids
Improvement in manufacture, particularly in the packaging of panty liners
Elimination of the turtle symbol (SUPD) on the packaging

### LIST OF REFERENCE SIGNS

1—test table
2—sample
3—slide
4—deflection pulley
5—load cell
6—direction of movement
7—polyamide cord
8—maximum force

1. A cover nonwoven for feminine hygiene products, consisting of a mechanically consolidated staple fiber nonwoven, which consists of 100% cellulose fiber materials
wherein surface of the cover nonwoven is mechanically smoothed,
the cover nonwoven has a static coefficient of friction of less than 0.20, and
the cover nonwoven has a dynamic coefficient of friction of less than 0.18.

2. The cover nonwoven of claim 1,
wherein the cellulose fiber materials are viscose or lyocell fibers.

3. The cover nonwoven of claim 1,
wherein the surface of the cover nonwoven is smoothed by using smooth calender rollers made of steel and/or rubber.

4. The cover nonwoven of claim 1,
wherein the cover nonwoven is consolidated using hydroentanglement.

5. A tampon for hygiene purposes consisting of a cover nonwoven, an absorbent core and a return thread,
wherein surface of the cover nonwoven is mechanically smoothed,
the cover nonwoven has a static coefficient of friction of less than 0.20, and
the cover nonwoven has a dynamic coefficient of friction of less than 0.18.

6. A panty liner or sanitary towel containing a cover nonwoven
wherein surface of the cover nonwoven is mechanically smoothed,
the cover nonwoven has a static coefficient of friction of less than 0.20, and
the cover nonwoven has a dynamic coefficient of friction of less than 0.18.
